SEQUENCE LISTING (6 sequences)

SEQ ID NO: 1 - DNA, 23 bp, Lactococcus lactis
ccttaggtattcgtatggttgac

SEQ ID NO: 2 - DNA, 18 bp, Lactococcus lactis
acccgcttgaacagagta

SEQ ID NO: 3 - DNA, 25 bp, Lactococcus lactis
caaccactccaccagttacgctgct

SEQ ID NO: 4 - DNA, 18 bp, Streptococcus pyogenes
ggcttcttccgtcttgac

SEQ ID NO: 5 - DNA, 20 bp, Streptococcus pyogenes
cctacaacagcactttggta

SEQ ID NO: 6 - DNA, 21 bp, Streptococcus pyognenes
cgccgccaccagtaccaagag

(19) United States
(12) Patent Application Publication
(10) Pub. No.: US 2015/0132741 A1
(43) Pub. Date: May 14, 2015

(54) EXTRACTION CONTROL FOR DNA

(71) Applicant: Vela Operation PTE. Ltd., Singapore (SG)

(72) Inventors: Ann Lee Ling, Singapore (SG); Xi Li, Singapore (SG)

(73) Assignee: VELA OPERATIONS PTE. LTD., Singapore (SG)

(21) Appl. No.: 14/402,584

(22) PCT Filed: May 17, 2013
(86) PCT No.: PCT/IB2013/054031

§ 371 Date: Nov. 20, 2014

(30) Foreign Application Priority Data
May 21, 2012 (GB) 1208926.4

(51) Int. Cl. C12Q 1/68

(52) U.S. Cl. CPC C12Q 1/6806
USPC 435/5; 435/6.12; 435/252.1

(57) ABSTRACT

The present invention relates to compounds for use in the control of extraction procedures, particular in connection with nucleic acid material for use in PCR and more preferably real time PCR (quantitative PCR), The extraction control according to the present invention is based on non-pathogenic bacterial material which can be produced at low cost, in large quantities and which has good stability.

This invention relates to the field of diagnostics, particularly to compounds serving as extraction controls in methods for the detection of the presence or absence of target nucleic acids, e.g. DNA, in a sample to be analyzed. Defined quantities of extraction controls of the invention are added to said samples and nucleic acids derived from such samples are subsequently analyzed in real-time PCR-based assays for the detection of target DNA. The present invention relates also to compositions, kits, assays, and articles of manufacture, comprising the extraction control of the invention as well as to methods for the extraction of nucleic acids and subsequent real-time PCR analysis, wherein the extraction controls are used.

### BACKGROUND ART

PCR is considered the most sensitive and rapid method for detecting nucleic acids of interest, e.g. nucleic acids derived from a pathogen in a particular sample. PCR is well known in the art and has been described in U.S. Pat. No. 4,683,195 to Mullis et al., U.S. Pat. No. 4,683,202 to Mullis, U.S. Pat. No. 5,298,392 to Atlas et al., and U.S. Pat. No. 5,437,990 to Burg et al.

For the PCR step, oligonucleotide primer pairs specific for each of the target nucleic acid are provided wherein each primer pair includes a first nucleotide sequence complementary to a sequence flanking the 5′ end of the target nucleic acid sequence and a second nucleotide sequence complementary to a nucleotide sequence flanking the 3′ end of the target nucleic acid sequence. The nucleotide sequences of each oligonucleotide primer pair are specific to particular nucleic acid target and should not cross-react with other nucleic acids.

PCR is currently the method of choice for the detection of target DNA, e.g. nucleic acids whose presence or absence may be detected in diagnostic applications, for example in the diagnosis of a bacterial infection, the presence of biological contaminants in water, food, etc.

Real-time PCR or qPCR (quantitative qPCR, which has been described in various textbooks, e.g., in Logan J, Edwards K, Saunders N (editors), (2009). Real-Time PCR: Current Technology and Applications. Caister Academic Press), can be used in the detection of DNA. When PCR is used in diagnostic applications, the accuracy of a diagnosis not only depends on the integrity of chemical compounds used for the amplification of target DNA sequences, but also on the availability of DNA successfully extracted from a sample.

The analysis of biological specimens, e.g. blood samples, stool samples, contaminated water, food, etc., can be influenced by diverse chemical and physical factors that can affect the integrity of nucleic acids present in such samples. Examples for such factors are those playing a role in storage and retrieval conditions of samples (temperature, pH, time that has elapsed between obtaining the sample and extracting the nucleic acids therein, etc.). Moreover, nucleic acids may be affected by the presence of potentially interfering factors in a sample, e.g. inhibitory proteins or nucleic acid degrading enzymes (DNAses, etc.). Failure to extract intact or sufficient amounts of DNA from a sample may also be due to had quality of reagents used in the extraction procedure.

One or more factors exerting a negative influence on the result of the extraction of target nucleic acids originally present in a sample may be responsible for failure of detection. When PCR is performed using DNA extracted from a sample, failure to amplify a target sequence may be incorrectly interpreted as absence of the target. For diagnostic applications, e.g., in the detection of pathogen-derived DNA or of oncogenes, it is therefore of utmost importance to include controls for the reliability of the DNA extraction procedure. In the absence of appropriate extraction controls, failure to detect a target sequence may be interpreted as false negative test result. This may have serious consequences as the results may the cause for an incorrect diagnosis and subsequently the wrong treatment of the source organism, e.g. a human patient.

It is important also that extraction controls have a good long-term storage stability (i.e. that the extraction control can be used for at least one year, preferably two years or longer after shipping) at relatively unfavorable conditions, e.g. elevated temperatures such as temperatures above 4° C., preferably above 20° C., and that the extraction controls can be subjected to harsh extraction conditions without fear of their degradation. Extraction controls should also be safe under laboratory conditions and represent no threat to environment or health of personnel working herewith.

The present invention relates to such improved extraction controls in methods aiming at detecting the presence or absence of target DNA in various sources. In particular, the present invention relates to the use of DNA derived from non-pathogenic bacteria, such as those belonging to the genus Lactococcus, preferably L. lactis, more preferably L. lactis subspecies cremoris as source of control DNA, and as an extraction control reagent for real-time PCR-based detection methods.

L. lactis is generally considered not pathogenic to animals, in particular mammals such as humans, plants or microorganisms although there are occasional reports on infections with these bacteria. L. lactis is a spherical, Gram-positive bacterium extensively used in the food industry. It is non-sporulating and non-motile and is regarded as a GRAS (generally regarded as safe) food-grade bacterium. L. lactis proves to be a useful choice because of the genetic accessibility, ease of its production at industrial scale-up as well the ease of its handling.

In some embodiments of the present invention, the non-pathogenic bacteria have been UV-treated to kill them before use as extraction controls. This avoids any risk for persons involved in commercial applications, manufacturing, handling, packaging, and laboratory uses. At the same time risks for the environment are completely avoided.

In other embodiments of the invention, the non-pathogenic bacteria referred to above have not been UV-treated as the use of such bacteria is generally not a risk, but sensitivity of real-time PCR methods is better than with UV-treated bacteria.

The present inventors have surprisingly found that non-pathogenic bacteria, in particular of the genus Lactococcus, preferably L. lactis can serve as ideal extraction controls for DNA extraction methods from a sample. The extracted DNA can be used in real-time PCR (qPCR) reactions.

Lactobacillus, e.g., L. lactis, has also the advantage that it can be produced in large amounts at low cost. Routine measures in laboratories for the treatment of waste material avoid any risks of contamination of the environment with such extraction control material. Simple hygienic measures, for example, through pasteurization or autoclaving are sufficient to avoid any risk. In addition, the bacteria used in the context of the present invention may optionally be UV-treated before use, which destroys their capacity to proliferate.

### DESCRIPTION OF THE INVENTION

In a first embodiment, the present invention relates to processes or methods for the detection and/or quantification of at least one target nucleic acid in a biological sample, wherein the method comprises a nucleic acid extraction step in the presence of a non-pathogenic bacterium that is added to said sample before extraction is carried out. Herein below, the addition of extraction control of the present invention to a biological sample before extraction of DNA is also referred to as “spiking”.

In a preferred embodiment of the invention, the quantification and/or detection process of target DNA comprises real time PCR (also referred to as “quantitative PCR”, qPCR).

A biological sample may include a sample obtained from a water supply; sewer treatment area; a soil sample from a farming area; animal grazing area; waste disposal area; and/or a sample obtained from virtually any water source used by animals or humans for consumption, cleaning, or any other domestic or commercial use; or the like. In addition, a biological sample may comprise human or animal waste materials (e.g., stool), medical refuse (bandages and wound dressings), body fluid (urine, plasma, blood, mucus, etc), and/or the like. In some embodiments, the methods provide for the screening and/or testing of a biological specimen such as drinking water and/or bodies of water (such as a stream, river, or lake) from which drinking water is obtained.

In further embodiments of the invention, the extraction of DNA can be performed using any method known in the art (cf. “Molecular Cloning: A Laboratory Manual”, Third and Fourth Editions; Sambrook et al.) or using kits available on the market from various commercial sources such as Qiagen (Hilden, DE) or Macherey-Nagel (DE).

In further embodiments of the invention, the nonpathogenic bacterium belongs to the genus Lactococcus, preferably it is L. lactis, more preferably L. lactis subspecies cremoris.

In preferred embodiments of the invention, the target DNA is of viral, bacterial, fungal or parasitic origin. In other embodiments of the invention, target DNA is found in the genome of an organism from which the sample was obtained, e.g. DNA playing a role in certain diseases, etc.

In yet other embodiments of the invention, methods or processes of the invention are directed to the amplification of target nucleic acids comprising the steps:
a) obtaining a sample suspected to contain target DNA;
b) extracting DNA from said sample in the presence of a pre-defined amount of an extraction control of the present invention, wherein said extraction control is added to the sample suspected to contain at least one nucleic acid molecule of interest/to be analyzed prior to extraction of nucleic acids;
c) amplification of target sequences using at least one reaction mixture comprising primers specifically hybridizing with a target molecule and/or a primer pair specifically hybridizing with DNA derived from the extraction control and/or probes, wherein said primers and/or probes carry fluorescent moieties allowing their detection;
d) monitoring the amplification process and/or detecting and quantifying the amount of products;
e) optionally calculating the amount of amplification products derived from both, extraction control nucleic and target nucleic acids;
f) optionally deciding, based on the results obtained in the preceding steps, on the therapeutic treatment of a patient.

In the above methods or processes of the invention, a pre-defined amount of the non-pathogenic bacterium, e.g. a bacterium belonging to the genus Lactococcus, preferably L. lactis, more preferably L. lactis subspecies cremoris is added. The amount should be sufficient to give a Ct value of ˜30 in the real-time PCR assay. This corresponds roughly to 10⁴ to 10⁶ CFU of, e.g., L. lactis used per extraction reaction. The amount of DNA material extracted from this quantity of L. lactis is low enough not to affect the detection sensitivity of the target nucleic acid, yet is of a significant enough amount to entail its efficient detection.

The present invention relates also to compositions for use in the extraction of DNA comprising an extraction control, said extraction control comprising a non-pathogenic bacterium. Said bacterium is preferably selected from bacteria of the genus Lactococcus, such as L. lactis, more preferably it is L. lactis cremoris.

In yet additional embodiments, the invention is directed to kits comprising an extraction control as defined in the above sections. Preferably, the kits according to the present invention are diagnostic kits, wherein the diagnostic kits comprise components for performing real-time PCR. The kits according to the present invention comprise an extraction control having a storage stability of at least one year, preferably 2, 3, 4, 5, 6, 7, 8, 9 or 10 years at ambient temperature. In some embodiments of the invention, the kit components may be freeze-dried and may contain a buffer for reconstitution.

In the some processes and/or methods of the invention, in some compositions or kits or other products according to the invention, the non-pathogenic bacterium has optionally been inactivated, i.e. is no longer able to divide. In preferred embodiments, the inactivation is achieved by UV-treatment of the bacteria. UV-treatment is a physicochemical inactivation method that leaves the structure of proteins generally intact, but it crosslinks nucleic acids. Traditionally, UV-treatment is not used in methods for the analysis of nucleic acids, but for protein-based assays such as ELISA, Western Blot and other protein-based assays.

The present invention pertains also to an assay for the diagnosis of the presence or absence of a target DNA molecule. Said assay comprises an extraction control as defined in any of the previous sections.

Furthermore, the present invention relates to automated devices for the extraction of nucleic acids from a biological sample comprising a source, e.g. a compartment of the device containing the extraction control of the invention.

Commercial uses of the present methods include clinical diagnosis of a human specimen, veterinary diagnosis from an animal specimen, water quality testing from recreational or drinking water samples, food sample testing, and environmental testing from soil or other sample types as defined above.

Compositions, components of kits or other products of the invention may be prepared in lyophilized form and/or provided in one or more master mixes optionally comprising additional components, e.g., for the extraction of nucleic acids and/or for performing PCR.

In some aspects of the invention, the primers and/or probes of the invention can be labeled with a fluorescent moiety. Fluorescent moieties for use in real-time PCR detection are known to persons skilled in the art and are available from various commercial sources, e.g. from Life Technologies™ or other suppliers of ingredients for real-time PCR.

In addition, the methods and products of the present invention may include a positive internal control for the PCR as known in the art or as described in UK-application No. GB 1204776.7. The term “internal control” as used herein refers to a nucleic acid sequence that may be used to demonstrate that a PCR reaction is functioning to detect a target sequence.

In some aspects of the invention, there are provided articles of manufacture, or kits. Articles of manufacture can include fluorophoric moieties for labeling the primers or probes or the primers and probes are already labeled with donor and corresponding acceptor fluorescent moieties.

Amplification generally involve the use of a polymerase enzyme. Suitable enzymes are known in the art, e.g. Taq Polymerase, etc.

As used herein, the term “probe” or “detection probe” refers to an oligonucleotide that forms a hybrid structure with a target sequence contained in a molecule (i.e., a “target molecule”) in a sample undergoing analysis, due to complementarity of at least one sequence in the probe with the target sequence. The nucleotides of any particular probe may be deoxyribonucleotides, ribonucleotides, and/or synthetic nucleotide analogs.

The term “primer” or “amplification primer” refers to an oligonucleotide that is capable of acting as a point of initiation for the 5′ to 3′ synthesis of a primer extension product that is complementary to a nucleic acid strand. The primer extension product is synthesized in the presence of appropriate nucleotides and an agent for polymerization such as a DNA polymerase in an appropriate buffer and at a suitable temperature.

The most widely used target amplification procedure is PCR, first described for the amplification of DNA by Mullis et al. in U.S. Pat. No. 4,683,195 and Mullis in U.S. Pat. No. 4,683,202 and is well known to those of ordinary skill in the art.

The term “real-time PCR” refers to the detection of PCR products via a fluorescent signal generated by the coupling of a fluorogenic dye molecule and a quencher moiety to the same or different oligonucleotide substrates. Examples of commonly used probes are TAQMAN® probes, Molecular Beacon probes, SCORPION® probes, and SYBR® Green probes. Briefly, TAQMAN® probes, Molecular Beacons, and SCORPION® probes each have a fluorescent reporter dye (also called a “fluor”) attached to the 5′ end of the probes and a quencher moiety coupled to the 3′ end of the probes. In the unhybridized state, the proximity of the fluor and the quencher molecules prevents the detection of fluorescent signal from the probe; during PCR, when the polymerase replicates a template on which a probe is bound, the 5′-nuclease activity of the polymerase cleaves the probe thus, increasing fluorescence with each replication cycle. SYBR Green® probes bind double-stranded DNA and upon excitation emit light; thus as PCR product accumulates, fluorescence increases. In the context of the present invention, the use of TAQMAN® probes is preferred. When real-time PCR is used, it is possible to measure or determine also the quantity of the target nucleic acid in the sample. In the latter case, the real-time PCR is often referred to as qPCR.

The terms “fluorophore”, “fluorogenic dye”, “fluorescent dye” as used herein designate a functional group attached to a nucleic acid that will absorb energy of a specific wavelength and re-emit energy at a different, but equally specific, wavelength.

The terms “complementary” and “substantially complementary” refer to base pairing between nucleotides or nucleic acids, such as, for instance, between the two strands of a double-stranded DNA molecule or between an oligonucleotide primer and a primer binding site on a single-stranded nucleic acid to be sequenced or amplified. Complementary nucleotides are, generally, A and T (or A and U), and G and C. Within the context of the present invention, it is to be understood that the specific sequence lengths listed are illustrative and not limiting and that sequences covering the same map positions, but having slightly fewer or greater numbers of bases are deemed to be equivalents of the sequences and fall within the scope of the invention, provided they will hybridize to the same positions on the target as the listed sequences. Because it is understood that nucleic acids do not require complete complementarity in order to hybridize, the probe and primer sequences disclosed herein may be modified to some extent without loss of utility as specific primers and probes. Generally, sequences having homology of about 90% or more fall within the scope of the present invention. As is known in the art, hybridization of complementary and partially complementary nucleic acid sequences may be obtained by adjustment of the hybridization conditions to increase or decrease stringency, i.e., by adjustment of hybridization temperature or salt content of the buffer.

The term “hybridizing conditions” is intended to mean those conditions of time, temperature, and pH, and the necessary amounts and concentrations of reactants and reagents, sufficient to allow at least a portion of complementary sequences to anneal with each other. As is well known in the art, the time, temperature, and pH conditions required to accomplish hybridization depend on the size of the oligonucleotide probe or primer to be hybridized, the degree of complementarity between the oligonucleotide probe or primer and the target, and the presence of other materials in the hybridization reaction admixture. The actual conditions necessary for each hybridization step are well known in the art or can be determined without undue experimentation.

The term “label” as used herein refers to any atom or molecule that can be used to provide a detectable (preferably quantifiable) signal, and that can be attached to a nucleic acid or protein via a covalent bond or noncovalent interaction (e.g., through ionic or hydrogen bonding, or via immobilization, adsorption, or the like). Labels generally provide signals detectable by fluorescence, chemiluminescence, radioactivity, colorimetry, mass spectrometry, X-ray diffraction or absorption, magnetism, enzymatic activity, or the like. Examples of labels include fluorophores, chromophores, radioactive atoms, electron-dense reagents, enzymes, and ligands having specific binding partners.

In another embodiment of the invention, the extraction control of the present invention is found in a compartment of a device that is suitable in fully automated laboratories capable of extracting nucleic acids from a sample, setting up amplification reactions, and performing said amplification reactions (e.g. qPCR) using the components described herein and quantitatively and/or qualitatively detecting nucleic acid targets, e.g. using real-time PCR.

In a further aspect, the present invention relates to a composition comprising primers and probes. Preferably, the composition comprises also ingredients, e.g. enzymes, buffers and nucleotides necessary for PCR, preferably for qualitative and/or quantitative PCR. The composition may be stored in the refrigerator in a liquid state or deep-frozen in a suitable medium, or it may be lyophilized and reconstituted before use and may further comprise detectable probes and/or an internal (positive) control.

The term “amplification” of DNA as used herein means the use of PCR to increase the concentration of a particular nucleic acid sequence within a mixture of nucleic acid sequences. The term “PCR” as used herein means the polymerase chain reaction, as is well-known in the art. The term includes all forms of PCR, such as, e.g., real-time PCR (quantitative PCR).

The particular nucleic acid sequence that is amplified is described herein as a “target” sequence. The term “target” sequence as used herein means the sequence of a nucleic acid that is amplified by PCR. The term “target” nucleic acid sequence as used herein means the sequence of a nucleic acid that is amplified by PCR.

The terms “biological sample” and “sample” as used herein mean any specimen or sample of matter capable of containing an organism. Non-limiting examples include a sample of water, a soil sample, an air sample, a stool sample, a urine sample, and the like. In other embodiments of the invention, the sample is a tissue fluid from a patient, which may be selected from the group consisting of blood, plasma, serum, lymphatic fluid, synovial fluid, cerebrospinal fluid, amniotic fluid, amniotic cord blood, tears, saliva, and nasopharyngeal washes.

The term “patient” as used herein is meant to include both human and veterinary patients.

The terms “pathogen,” “organism,” and “species” are used interchangeably herein and refer to any one species, or closely-related group of species, that may be uniquely identified by an oligonucleotide sequence. The species may be known or unknown and may include any type of virus, bacterium, fungus, etc.

The term “primer pair” as used herein means a pair of oligonucleotide primers that are complementary to the sequences flanking a target sequence. The primer pair consists of a forward primer and a reverse primer. The forward primer has a nucleic acid sequence that is complementary to a sequence upstream, i.e. 5′ of the target sequence. The reverse primer has a nucleic acid sequence that is complementary to a sequence downstream, i.e. 3′ of the target sequence.

The terms “probe” and “probe pair” refer to one or two oligonucleotide sequences that are complementary to a specific target sequence and are covalently linked to a fluorophore. A probe pair includes two oligonucleotides: a “donor probe” and an “acceptor probe.” When both probes are bound to the target sequence, the donor probe's fluorophore may transfer energy to the acceptor probe's fluorophore in a Förster resonance energy transfer (FRET).

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention.

It also is be noted that as used herein and in the appended claims, the singular forms “a,” “an,” and “the” include the plural reference unless the context clearly dictates otherwise. This, for example, a reference to “a capsule” is a reference to one or more capsules and equivalents thereof known to those skilled in the art.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein.

Any numerical values recited herein include all values from the lower value to the upper value in increments of one unit provided that there is a separation of at least two units between any lower value and any higher value. As an example, if it is stated that the concentration of a component or value of a process variable such as, for example, size, temperature, pressure, time and the like, is, for example, from 1 to 90, specifically from 20 to 80, more specifically from 30 to 70, it is intended that values such as 16 to 85, 22 to 68, 43 to 51, 30 to 32 etc., are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

Particular methods, devices, and materials are described, although any methods and materials similar or equivalent to those described herein can be used in the practice or testing of the invention. All references referred to herein are incorporated by reference herein in their entirety.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In addition, the materials, methods, and examples are illustrative only and not intended to be limiting. All publications, patent applications, patents, and other references mentioned herein are incorporated by reference in their entirety. In case of conflict, the present specification, including definitions, will be decisive.

The details of one or more embodiments of the invention are set forth in the description below. Other features, objects, and advantages of the invention will be apparent from the description, and from the claims.

### EXAMPLES

### Primers and Probes Design (5′ to 3′) Specific for Lactococcus lactis

| | |
|---|---|
| Forward | CCTTAGGTATTCGTATGGTTGAC |
| primer | (SEQ ID NO: 1) |
| Reverse | ACCCGCTTGAACAGAGTA |
| primer | (SEQ ID NO: 2) |
| Probe | Quasar670-CAACCACTCCACC |
| | AGTTACGC-BHQ2 |
| | (SEQ ID NO. 3) |

### Experimental Data

Amplification of Streptococcus pyogenes genomic DNA by specific primers and probes.
L. lactis was subjected to UV killing. 100% killing was achieved as tested by spread plating. From the resulting bacteria, 100 μl of the following dilutions was used for DNA extraction using the Qiagen DNA extraction kit and eluted with 100 μl AE buffer.
A real-time PCR assay was run with the following primer-probe combinations, using Streptococcus pyogenes genomic DNA as template.

| Primer | |
|---|---|
| combination | Primer sequence |
| S. pyogenes | GGCTTCTTCCGTCTTGAC (SEQ ID NO: 4) |
| forward | |
| S. pyogenes | CCTACAACAGCACTTTGGTA (SEQ ID NO: 5) |
| reverse | |
| S. pyogenes | FAM-CGCCGCCACCAGTACCAAGAG-BHQ1 |
| probe | (SEQ ID NO: 6) |

S. pyogenes Taqman probes are labeled with FAM reporter and BHQ-1 quencher. L. lactis extraction control (EC) was added to samples during prior to DNA extraction.

A PCR reaction was set-up according to the parameters below.

| | Volumes/final concentration |
|---|---|
| Component | for 1 reaction |
| Roche FastStart mastermix (2x) | 12.5 μl |
| S. pyogenes forward primer | 0.5 μM final concentration |
| S. pyogenes reverse primer | 0.5 μM final concentration |
| S. pyogenes probe | 0.25 μM final concentration |
| S. pyogenes gDNA | 10 pg |
| L. lactis forward primer | 0.5 μM final concentration |
| L. lactis reverse primer | 0.5 μM final concentration |
| L. lactis probe | 0.25 μM final concentration |
| L. lactis gDNA | 10²-10⁸ CFU |
| H₂O | Top up to 25 μl final volume |
| Total Volume | 25 μl |

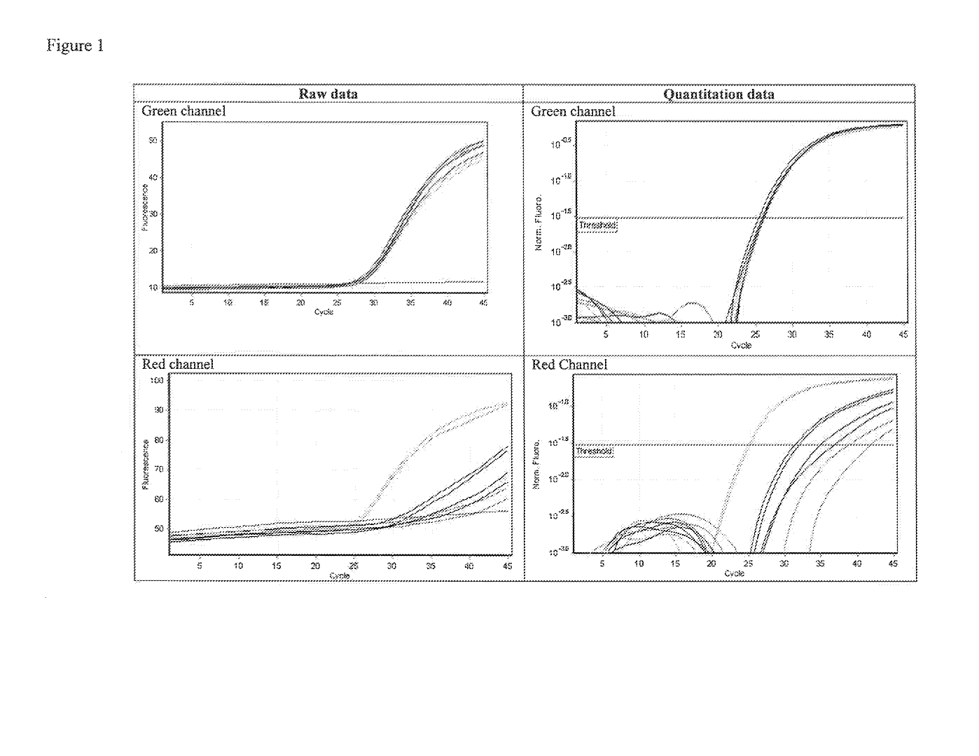

A Rotor-gene Q machine (Qiagen) was used and PCR reactions were run with the following cycling conditions: 95° C. for 10 min followed by 45 cycles at 95° C. for 15 seconds and 60° C. for 60 seconds. S. pyogenes was detected in the green channel, whereas the EC L. lactis was detected in the red channel (cf. FIG. 1). The graphs on top of FIG. 1 show the results of real-time PCR specific for S. pyogenes and the two graphs on the bottom of FIG. 1 show the results obtained with the extraction control. Each reaction was performed in duplicate and the results are listed in the table below.

| EC L. lactis | | | | |
|---|---|---|---|---|
| concentration | EC L. lactis (red channel) | | S. pyogenes (green channel) | |
| (CFU) | Ct | Average Ct | Ct | Average Ct |
| 10⁸ | 25.21 | 25.08 | 25.98 | 25.94 |
| | 24.94 | | 25.91 | |
| 10⁴ | 31.99 | 31.69 | 25.85 | 25.95 |
| | 31.38 | | 26.05 | |
| 10³ | 41.84 | 40.34 | 26.21 | 26.21 |
| | 38.84 | | 26.21 | |
| 10² | 36.86 | 35.93 | 25.39 | 25.73 |
| | 35.00 | | 26.06 | |

The assay would be able to rapidly diagnose clinical samples for the presence of S. pyogenes based on the S. pyogenes exotoxin B (SpeB) gene.

This assay is designed as a duplex assay. The advantage of this is it allows L. lactis to be added to clinical samples. L. lactis DNA could serve as an internal extraction control to show that DNA is successfully extracted. When a real-time PCR reaction provides negative signals for both S. pyogenes and L. lactis, this could mean that DNA extraction was unsuccessful and this eliminates a false negative reading.

As shown in the data, the presence of L. lactis DNA and L. lactis primer/probes does not affect the sensitivity of the detection of 10 pg of S. pyogenes DNA.

1. A method for the detection and/or quantification of target DNA in a sample, said method comprising
a) extracting DNA from said sample in the presence of a non-pathogenic bacterium, wherein said non-pathogenic bacterium is added to said sample prior to extracting;
b) amplifying the extracted DNA; and
c) detecting and/or quantifying the target DNA.

2. The method according to claim 1, wherein a defined amount of non-pathogenic bacterium is added to the sample.

3. The method according to claim 1, wherein the non-pathogenic bacterium belongs to the genus Lactococcus.

4. The method according to claim 3, wherein the non-pathogenic bacterium is Lactococcus lactis.

5. The method according to claim 4, wherein the non-pathogenic bacterium is Lactococcus lactis subspecies cremoris.

6. The method according to claim 1, wherein the amplification step comprises PCR.

7. The method according to claim 6, wherein the amplification step is real-time PCR.

8. The method according to claim 1, wherein the target DNA is pathogen-derived DNA or DNA derived from the donor of the sample.

9. The method according to claim 8, wherein the pathogen-derived DNA is of viral, bacterial, fungal or parasitic origin.

10. A method for the amplification of target nucleic acids comprising:
a) obtaining a sample suspected to contain said target nucleic acid;
b) extracting nucleic acids from said sample in the presence of a non-pathogenic bacterium as an extraction control, wherein said extraction control is added to the sample suspected to contain at least one of said target nucleic acids prior to extracting said nucleic acids;
c) amplifying said target nucleic acids using an amplification reaction mixture comprising primers specifically hybridizing with said target nucleic acids and a primer pair specifically hybridizing with nucleic acids derived from the extraction control;
d) quantifying the amplification products.

11. The method of claim 10, comprising real-time PCR.

12. The method of claim 10, wherein said non-pathogenic bacterium belongs to the genus Lactococcus.

13. The method according to claim 10, wherein said target nucleic acid is pathogen-derived DNA or DNA derived from the donor of the sample.

14. A composition comprising the extraction control of claim 10.

15. The composition according to claim 14, wherein the extraction control is a non-pathogenic bacterium which belongs to the genus Lactococcus.

16. A kit comprising the extraction control of claim 10.

17. The kit according to claim 16, wherein the kit is a diagnostic kit.

18. The kit according to claim 17, wherein the diagnostic kit is for PCR or real-time PCR.

19. The kit according to claim 16, wherein the kit further comprises reagents for the extraction of nucleic acids.

20. The kit according to claim 16, wherein the extraction control has a storage stability of at least one year at ambient temperature.

21. The method of claim 1 wherein the non-pathogenic bacterium has been inactivated.

22. The kit according to claim 16, wherein the components are freeze-dried and contain a buffer for reconstitution.

23. The method of claim 10 wherein the non-pathogenic bacterium has been treated with UV.

24. An assay for the diagnosis of the presence or absence of a target nucleic acid molecule comprising the extraction control of claim 10.

25. A device for the extraction of DNA comprising a DNA extraction control.

26. The device according to claim 25, wherein said extraction control is a non-pathogenic bacterium.

27. (canceled)

28. (canceled)

29. (canceled)

30. The composition according to claim 14 wherein the non-pathogenic bacterium has been inactivated.

31. The kit according to claim 16 wherein the non-pathogenic bacterium has been inactivated.

32. The composition according to claim 14 wherein the non-pathogenic bacterium has been treated with UV.

33. The kit according to claim 16 wherein the non-pathogenic bacterium has been treated with UV.

34. The method of claim 10 wherein the non-pathogenic bacterium is Lactococcus lactis.

35. The method of claim 34 wherein the non-pathogenic bacterium is Lactococcus lactis subspecies cremoris.

36. The method of claim 13 wherein the pathogen-derived DNA is of viral, bacterial, fungal or parasitic origin.

37. The composition of claim 15 wherein the non-pathogenic bacterium is Lactococcus lactis.

38. The composition of claim 37 wherein the non-pathogenic bacterium is Lactococcus lactis subspecies cremoris.
